(19) United States
(12) Patent Application Publication
(10) Pub. No.: US 2005/0004441 A1
(43) Pub. Date: Jan. 6, 2005

(54) System and methods for locating and ablating arrhythomogenic tissues

(76) Inventors: Peter C. Chen, Irvine, CA (US); Alan de la Rama, Cerritos, CA (US); Cary K. Hata, Tustin, CA (US); Vivian Tran, Santa Ana, CA (US)

Correspondence Address: Raymond Sun;Law Offices of Raymond Sun, 12420 Woodhall Way, Tustin, CA, 92782, US

(21) Appl. No.: 10/897,887

(22) Filed: Jul. 22, 2004

Related U.S. Application Data
(63) Continuation-in-part of application No. 10/744,354, filed on Dec. 22, 2003.
Application No. 10/744,354 is a continuation of application No. 09/975,269, filed on Oct. 11, 2001, now Pat. No. 6,671,533.

(51) Int. Cl. A61B 18/04

(52) U.S. Cl. 600/374; 606/27

(57) ABSTRACT

A catheter for sensing electrical events about a selected annulus region of the heart and for treating tissue in the selected annulus region has a handle assembly, and a shaft having a proximal end coupled to the handle assembly. The catheter also has a mapping element provided adjacent its distal end, and an ablation element positioned spaced apart along the shaft from the mapping element. The mapping element is first positioned distally to the desired treatment location in the selected annulus region and the distal location is mapped. The expandable member enclosing the ablation element is inflated and contrast medium injected to determine the orientation of the ablation element with respect to the annulus region. After the target ablation site is determined and the PV potentials verified, the ablation element is activated for therapeutic energy delivery.

### BACKGROUND OF THE INVENTION

1. Related Cases

This is a continuation-in-part of co-pending Ser. No. 10/744,354, entitled “System and Method for Mapping and Ablating Body Tissue of the Interior Region of the Heart”, filed Dec. 22, 2003, which is in turn a continuation of Ser. No. 09/975,269, filed Oct. 11, 2001, now U.S. Pat. No. 6,671,533, whose disclosures are incorporated by this reference as though fully set forth herein.

2. Field of the Invention

The present invention is directed to systems and methods for mapping and ablating body tissue of the interior regions of the heart for treating cardiac arrrhythmias.

3. Description of the Prior Art

Atrial fibrillation (AF) is a common cardiac arrhythmia associated with significant morbidity and mortality. A number of clinical conditions may arise from irregular cardiac functions and the resulting hemodynamic abnormalities associated with AF, including stroke, heart failure and other thromboembolic events. AF is a significant cause of cerebral stroke, wherein the fibrillating motion in the left atrium induces the formation of thrombus. A thromboembolism is subsequently dislodged into the left ventricle and enters the cerebral circulation where stroke may result.

For many years, the only curative treatment for AF has been surgical, with extensive atrial incisions used to compartmentalize the atrial mass below that critical for perpetuating AF. Recently, transcatheter linear radiofrequency ablation in the right or left atrium has been used to replicate surgical procedures in patients with paroxysmal or chronic AF. Such ablation is carried out by a catheter system that performs both mapping and ablation. With current techniques, there is still uncertainty regarding the number of lesions, the optimum ablation site, and the need for continuous lines. As a result, focal ablation has been proposed as an alternative approach, due to the belief that ectopic beats originating within or at the ostium of the pulmonary veins (PV) may be the source of paroxysmal and even persistent AF. Although successful, the technical feasibility of this technique is restricted by the difficulty in mapping the focus if the patient is in AF or has no consistent firing, the frequent existence of multiple foci causing high recurrence rates, and a high incidence of PV stenosis.

However, there are a number of drawbacks associated with the catheter-based mapping and ablation systems that are currently known in the art. One serious drawback lies in the unstable positioning of the catheter inside the atrium of the heart. When a catheter is not properly stabilized, the mapping becomes difficult and inaccurate.

Another drawback is associated with certain catheter-based systems that utilize an expandable balloon that is inflated to conform to the pulmonary vein ostium. After the balloon is inflated and the catheter positioned, it becomes difficult to map or record the distal PV potentials without removing this catheter and placing another mapping catheter inside the PV. Moreover, inflation of the balloon to conform to the pulmonary vein ostium blocks blood flow to the left atrium, and such prolonged blockage can have adverse effects to the patient. Blockage of blood flow from the PV deprives the patient from receiving oxygenated blood. In addition, the blockage may be a potential source for stenosis.

Thus, there still remains a need for a catheter-based system and method that can effectively map and ablate potentials (also known as spikes) inside PVs which can induce paroxysmal AF, while avoiding the drawbacks set forth above.

### SUMMARY OF THE DISCLOSURE

It is an objective of the present invention to provide a system and method that effectively maps or records distal PV potentials and ablates the PV ostium.

It is another objective of the present invention to provide a system and method that effectively maps and ablates potentials without blocking blood flow.

In order to accomplish the objects of the present invention, there is provided a catheter for sensing electrical events about a selected annulus region of the heart and for treating tissue in the selected annulus region. The catheter has a handle assembly, and a shaft having a proximal end coupled to the handle assembly, a mapping element provided adjacent its distal end, and an ablation element positioned spaced apart along the shaft from the mapping element. The mapping element is first positioned distally to the desired treatment location in the selected annulus region and the distal location is mapped. The expandable balloon enclosing the ablation element is inflated and contrast medium injected to determine the orientation of the ablation element with respect to the annulus region. After the target ablation site is determined and the PV potentials verified, the ablation element is activated for therapeutic energy delivery.

### BRIEF DESCRIPTION OF THE DRAWINGS

FIG. 1 illustrates a mapping and ablation system according to one embodiment of the present invention.

FIG. 2 is a perspective view of the catheter of the system of FIG. 1.

FIG. 3 is an enlarged view of the distal tip section of the catheter of FIGS. 1 and 2.

FIG. 4 is a cross-sectional view of the distal tip section of FIG. 3 taken along lines A-A thereof.

FIG. 5 is a cross-sectional view of the distal tip section of FIG. 3 taken along lines B-B thereof.

FIG. 6 illustrates how the catheter of FIGS. 1 and 2 is deployed for use inside the heart of a patient.

FIG. 7 is a cross-sectional view illustrating the catheter of FIGS. 1 and 2 in use in a pulmonary vein during the mapping and ablation steps.

FIG. 8 illustrates the steering mechanism of the catheter of FIGS. 1 and 2.

FIG. 9 illustrates a mapping and ablation system according to another embodiment of the present invention.

FIG. 10 is a perspective view of the catheter of the system of FIG. 9.

FIG. 11 is an enlarged view of the distal tip section of the catheter of FIGS. 9 and 10.

FIG. 12 is a cross-sectional view of the distal tip section of FIG. 11 taken along lines A-A thereof.

FIG. 13 is a cross-sectional view of the distal tip section of FIG. 11 taken along lines B-B thereof.

FIG. 14 is an enlarged persepective view of the distal tip section of the catheter of FIGS. 9 and 10.

FIG. 15 illustrates a mapping and ablation system according to another embodiment of the present invention.

FIG. 16 is an enlarged persepective view of the distal tip section of the catheter of FIG. 15.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims. In certain instances, detailed descriptions of well-known devices, compositions, components, mechanisms and methods are omitted so as to not obscure the description of the present invention with unnecessary detail.

The present invention provides a catheter system that has two separate elements for performing the mapping and ablation operations. A first element that includes ring electrodes is provided along a distal ring and functions to map the region of the heart that is to be treated. After the mapping has been completed, a second element that includes a transducer mounted inside a balloon is positioned at the location where ablation is to be performed, and is used to ablate the selected tissue. During the ablation, the distal ring functions to anchor the position of the balloon, while the balloon is inflated to a diameter that is less than the diameter of the distal ring and the annulus where the treatment is taking place. As a result, blood can still flow unimpeded through the annulus.

Even though the present invention will be described hereinafter in connection with treating AF, it is understood that the principles of the present invention are not so limited, but can be used in other applications (e.g., treatment of accessory pathways, atrial flutter, ventricular tachycardia), and in other body pathways (e.g., right atrium, superior vena cava, right ventricle, left ventricle).

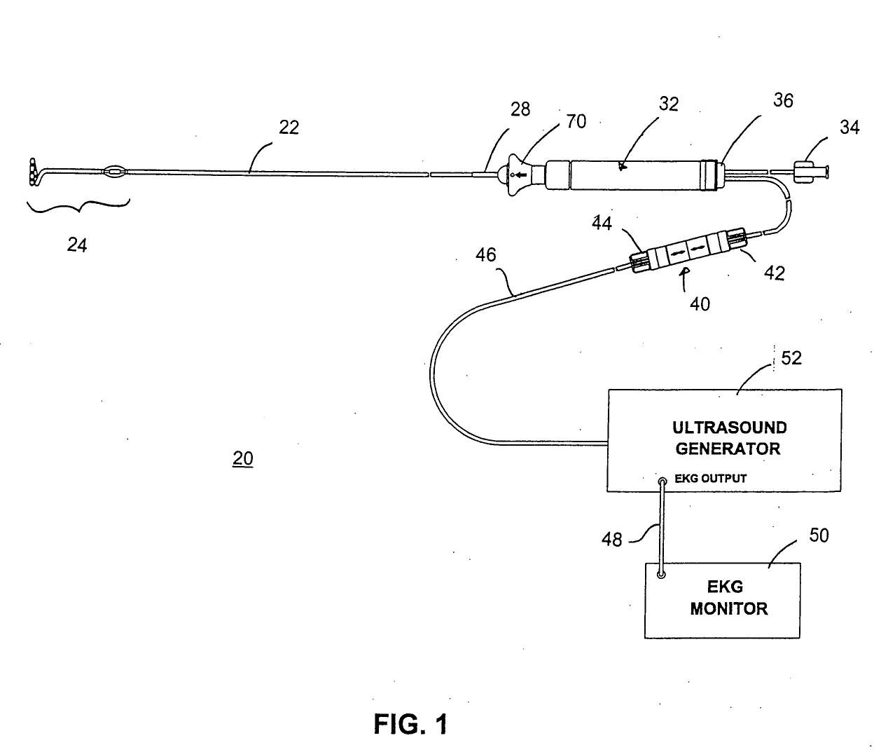

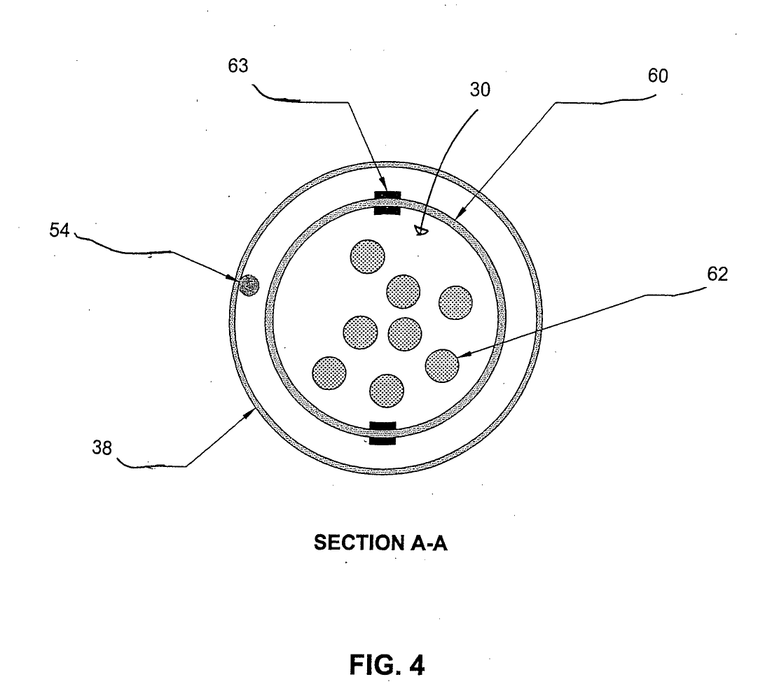

FIGS. 1-8 illustrate a catheter system 20 according to one embodiment of the present invention. The catheter system 20 has a tubular shaft 22 having a distal tip section 24, a distal end 26, a proximal end 28, and at least one lumen 30 extending through the shaft 22. A handle assembly 32 is attached to the proximal end 28 of the shaft 22 using techniques that are well-known in the catheter art.

The distal tip section 24 includes an expandable balloon 38 and a distal ring 80 that makes up the distal-most end of the shaft 22. A transducer 60 (e.g., piezoelectric or ultrasound) is housed inside the balloon 38. The balloon 38 can be made from any conventional material (such as but not limited to silicone, polyurethane, latex, polyamide and polyethylene), and heat bonded or otherwise attached to the shaft 22 using techniques that are well-known in the catheter art.

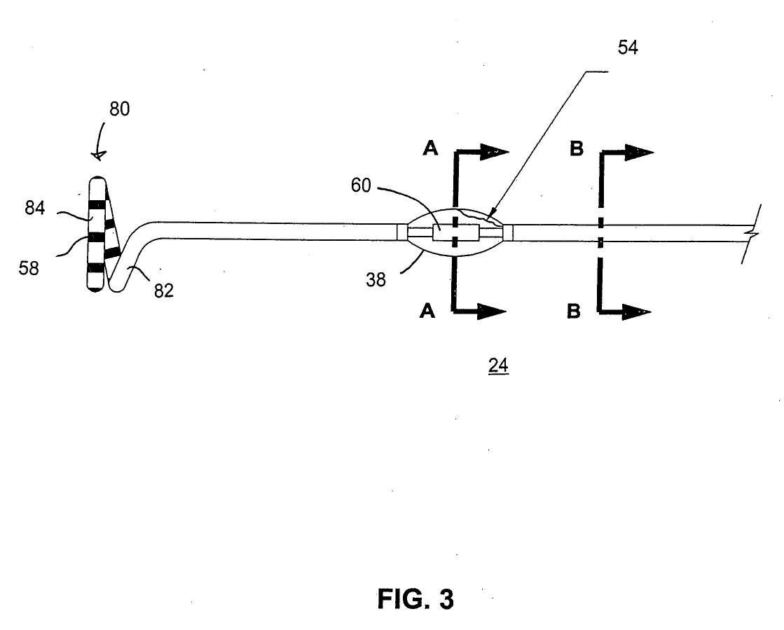

The distal ring 80 can be preformed into a generally curved or circular shape, resembling an open loop. The shape of the distal ring 80 corresponds to the circumferential geometry of a selected annulus (e.g., the PV) in the heart. In fact, the preformed shape of the distal ring 80 can be provided in a variety of curved geometries to overlie the anatomical geometry of the selected annulus. The distal ring 80 includes a transition section 82 that extends distally at an angle from the longitudinal axis of the shaft 22, and has a generally open-looped circular section 84 that extends from the transition section 82. As best seen from FIG. 3, the circular section 84 is oriented at an approximately perpendicular orientation from the longitudinal orientation of the shaft 22. The distal ring 80 can be made from the same material as the shaft 22. Such a material can be an electrically nonconductive, biocompatible, resilient plastic material which retains its shape and which does not soften significantly at human body temperature (e.g., Pebax™, polyethylene or polyester). As a non-limiting example, the geometry of the distal ring 80 can be created by thermoforming it into the desired shape.

A plurality of thermocouple wires 54 can have their distal tips secured to the interior surface of the balloon 38 (see FIG. 3), and are used to detect the temperature at the treatment site.

A plurality of ring electrodes 58 are provided in spaced-apart manner about the circular section 84 of the distal ring 80. The ring electrodes 58 can be made of a solid, electrically conducting material, like platinum or gold, that is attached about the circular section 84. Alternatively, the ring electrodes 58 can be formed by coating the exterior surface of the circular section 84 with an electrically conducting material, such as platinum or gold. The coating can be applied by sputtering, ion beam deposition or similar known techniques. The number of ring electrodes 58 can vary depending on the particular geometry of the region of use and the functionality desired.

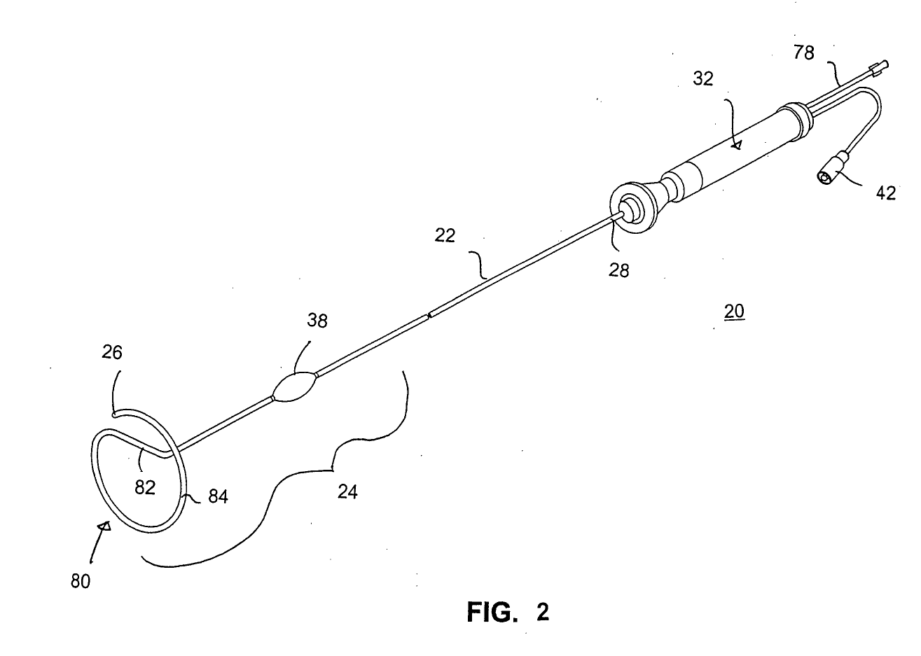

As will be explained in greater detail below, the ring electrodes 58 function to map the region of the heart that is to be treated. After the mapping has been completed, the balloon 38 is positioned at the location where ablation is to be performed, and the distal ring 80 functions to anchor the position of the balloon 38. The balloon 38 is expanded, but even the greatest expanded diameter of the balloon 38 will be provided to be less than the diameter of the distal ring 80 when the distal ring 80 is fully deployed (see FIGS. 2, 3 and 7). The ablation is then carried out by energy that is emitted from the ultrasound transducer 60 through the inflation media (e.g., fluid, saline, contrast media or mixture) inside the balloon 38, and the balloon 38 itself.

A standard Luer fitting 34 is connected to the proximal end 36 of the handle assembly 32 using techniques that are well-known in the catheter art. The Luer fitting 34 provides a fluid line for inflation media to be introduced to inflate the balloon 38 at the distal tip section 24 of the shaft 22. The inflation media is delivered via an inflation lumen 76 that extends from the handle assembly 32 (and coupled to the line 78 of the Luer fitting 34), and terminates at the balloon 38.

A connector assembly 40 is also connected to the proximal end 36 of the handle assembly 32 using techniques that are well-known in the catheter art. The connector assembly 40 has a proximal connector 42 that couples the handle assembly 32 to the connector 44 of a control line 46 that leads to an ultrasound generator 52. An EKG monitor 50 is coupled to the ultrasound generator 52 via another line 48. The EKG monitor 50 can be a conventional EKG monitor which receives (via the ultrasound generator 52) electrical signals detected by the ring electrodes 58 at the distal tip section 24, and processes and displays these electrical signals to assist the physician in locating the site of potentials in a PV. The ultrasound generator 52 can be a conventional ultrasound generator that creates and transmits ablating energy to the ultrasound transducer 60 that is positioned inside the balloon 38. The ultrasound transducer 60 will emit the energy to ablate the tissue that extends radially from the position of the balloon 38.

Electrical wires (not shown) extend from the ultrasound generator 52 along the lines 46 and 48, and conductor wires 62 and ultrasound wires 63 extend through the connector assembly 40, the handle assembly 32 and the lumen 30 of the shaft 22 to the distal tip section 24 of the shaft 22 to couple the ring electrodes 58 and the transducer 60, respectively. In addition, the thermocouple wires 54 can extend from the balloon 38 through the lumen 30 of the shaft 22 and the handle assembly 32 to the proximal connector 42, where they can be electrically coupled by the wires in the line 46 to the ultrasound generator 52 where the temperature can be displayed.

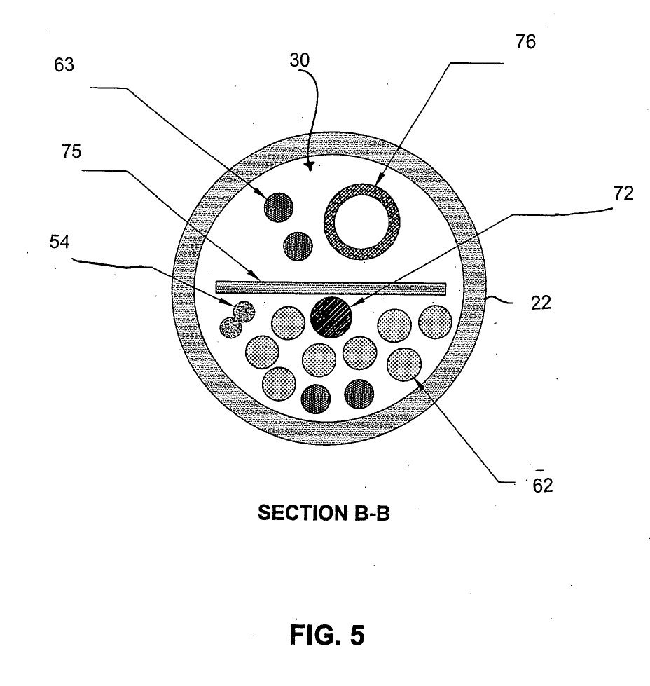

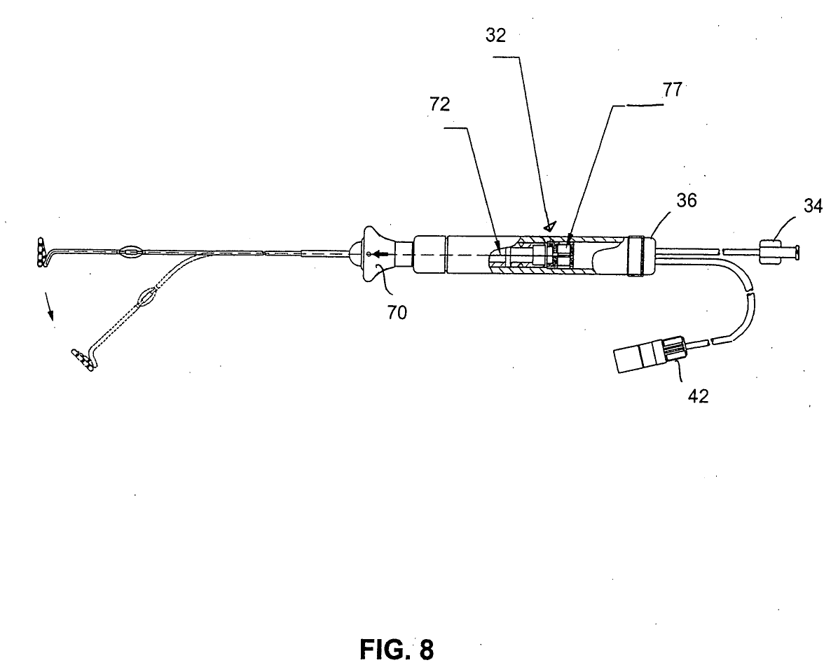

The handle assembly 32 also includes a steering mechanism 70 that functions to deflect the distal tip section 24 of the shaft 22 for maneuvering and positioning the distal tip section 24 at the desired location in the heart. Referring to FIGS. 1, 5 and 8, the steering mechanism 70 includes a steering wire 72 that extends in the main lumen 30 of the shaft 22 from its proximal end at the handle assembly 32 to its distal end which terminates in the distal tip section 24 before the location of the balloon 38. The proximal end of the steering wire 72 is wound around or secured to an anchor 77 that is fixedly positioned inside the handle assembly 32. The steering mechanism 70 also includes a flat wire 75 that extends in the lumen 30 from the anchor 77 to its distal end at a location slightly proximal to the balloon 38 (as shown in FIG. 5). The flat wire 75 is attached to the steering wire 72 at the distal ends of the flat wire 75 and the steering wire 72 so as to be controlled by the steering wire 72. Specifically, by pushing the steering mechanism 70 forward in a distal direction, the steering mechanism 70 will pull the steering wire 72 in a proximal direction, causing the distal tip section 24 to deflect to one direction (see in phantom in FIG. 8). By pulling back the steering mechanism 70 in a proximal direction, the steering wire 72 is deactivated and the distal tip section 24 returns to its neutral position or deflects to the opposite direction.

The distal ring 80 can be preformed to a fixed size (i.e., diameter) and shape that cannot be changed. Alternatively, the diameter of the distal ring 80 can be adjusted using techniques and incorporating mechanisms that are well-known in the catheter art.

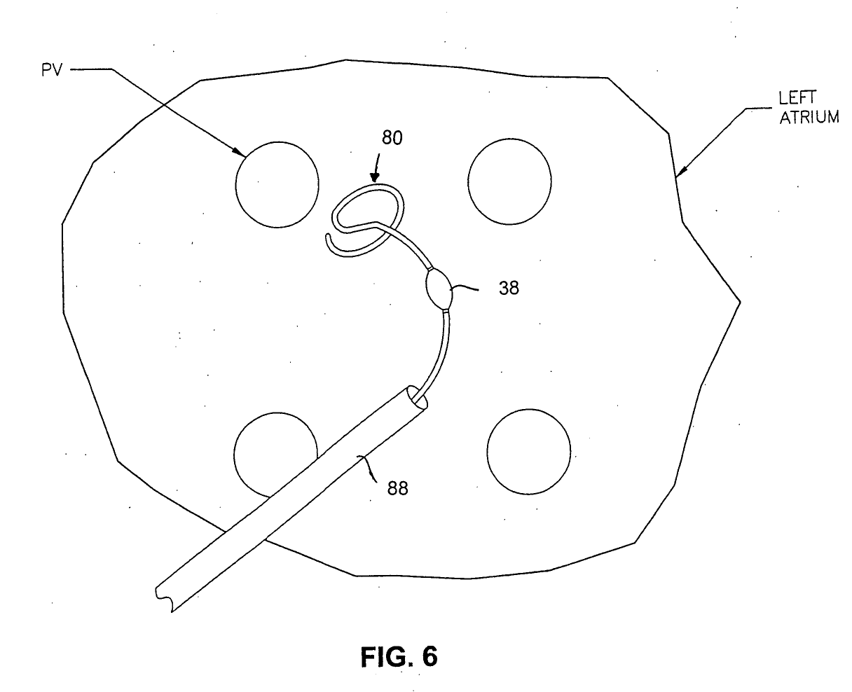

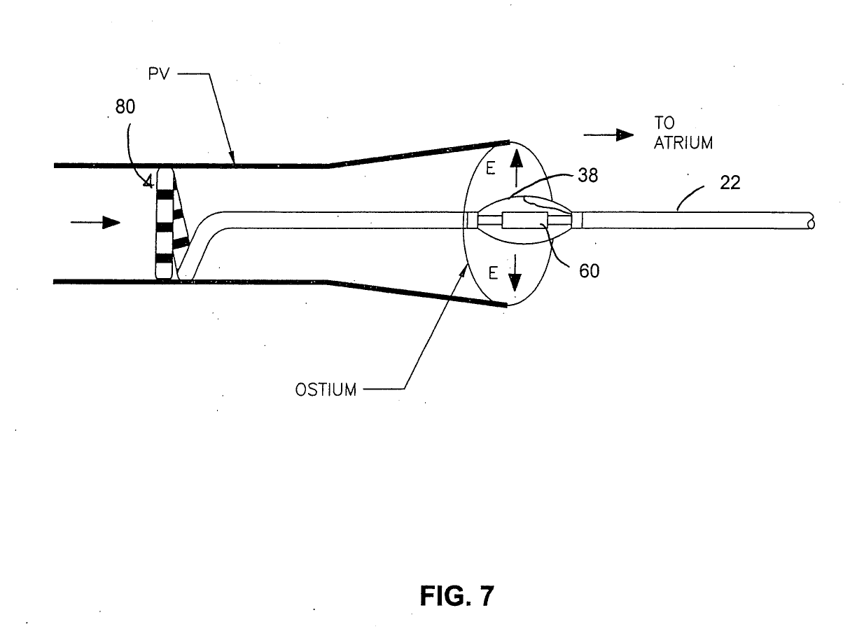

FIGS. 6 and 7 illustrate how the catheter system 20 is used. First, a guide sheath 88 is provided to deliver the shaft 22 and distal ring 80 to the desired location (e.g., the left atrium) in the heart. The shaft 22 is slid into the hollow lumen of the guide sheath 88, and the guide sheath 88 can slide forward and backward along the longitudinal axis of the shaft 22. When the guide sheath 88 is slid forwardly towards the distal ring 80, the distal ring 40 is progressively straightened out and drawn into the lumen of the guide sheath 88. Thus, when confined with the guide sheath 88, the distal ring 80 assumes the generally linear low profile shape of the guide sheath 88, which allows a physician to employ conventional percutaneous access techniques to introduce the catheter 20 into a selected region of the heart through a vein or artery. When the guide sheath 88 is slid rearwardly away from the distal ring 80, the distal ring 80 is uncovered and its resilient memory will cause the distal ring 80 to re-assume its preformed generally circular shape.

To introduce and deploy the distal tip section 24 within the heart, the physician uses a conventional introducer to establish access to a selected artery or vein. With the guide sheath 88 confining the distal ring 80, and with the balloon 38 deflated, the physician introduces the shaft 22 and the guide sheath 88 through a conventional hemostatic valve on the introducer and progressively advances the guide sheath 88 through the access vein or artery into the desired atrium, such as the left atrium as shown in FIG. 6. The physician observes the progress of the guide sheath 88 using fluoroscopic or ultrasound imaging. The guide sheath 88 can include a radio-opaque compound, such as barium, for this purpose. Alternatively, radio-opaque markers can be placed at the distal end of the guide sheath 88.

The shaft 22 and the guide sheath 88 can be maneuvered to the left atrium by the steering mechanism 70. Once located in the left atrium, the physician slides the guide sheath 88 back to free the distal ring 80 which resiliently returns to its preformed shape. The distal ring 80 is then maneuvered into contact with the selected annulus (e.g., the ostium) with the aid of fluoroscopy. Good contact is established when the ring electrodes 58 contact the selected annulus, and at this time, the physician operates a control located on the ultrasound generator 52 to effectuate the mapping of the selected annulus by the ring electrodes 58. The results of the mapping operation are processed and displayed at the EKG monitor 50. A differential input amplifier (not shown) in the EKG monitor 50 processes the electrical signals received from the ring electrodes 58 via the wires 62, and converts them to graphic images that can be displayed. The thermocouple wires 54 can also function to monitor the temperature of the surrounding tissue, and provide temperature information to the ultrasound generator 52. Throughout this mapping operation, the balloon 38 remains deflated.

Once the mapping operation has been completed and the desired position of the balloon 38 has been confirmed, the physician can then inflate the balloon 38 using inflation media. The balloon 38 is preferably manufactured using known techniques to a predetermined diameter so that its diameter at its maximum expansion will be less than the diameter of the distal ring 80 and the annulus or vessel (e.g., the PV in FIG. 7) where the ablation is to take place. The physician then controls the ultrasound generator 52 to generate ultrasound energy that is propagated through the wires 63 to the ultrasound transducer 60 that is positioned inside the balloon 38. The energy radiates in a radial manner from the transducer 60, propagates through the inflation media (which acts as an energy transmitting medium) inside the balloon 38, exits the balloon 38 and then reaches the selected tissue (typically in a waveform) to ablate the tissue. See the arrows E in FIG. 7 which illustrate the radiation of the energy from the transducer 60.

During the ablation, the distal ring 80 functions to anchor the distal tip section 24 inside the PV at the desired location so that the ablation can be performed accurately. In contrast to known catheter systems where the same element is used to anchor and ablate, by providing a separate element (i.e., the distal ring 80) to anchor the distal tip section 24, the function of the ablation element (i.e., the balloon 38 and transducer 60) will not be affected by the anchoring device, thereby ensuring that the ablation is performed accurately and effectively. In addition, since the maximum diameter of the balloon 38 is always smaller than the smallest diameter of the distal ring 80, blood will be able flow through the distal ring 80 and around the surfaces of the balloon 38.

When the ablation has been completed, the balloon 38 is deflated and the distal tip section 24 withdrawn from the heart.

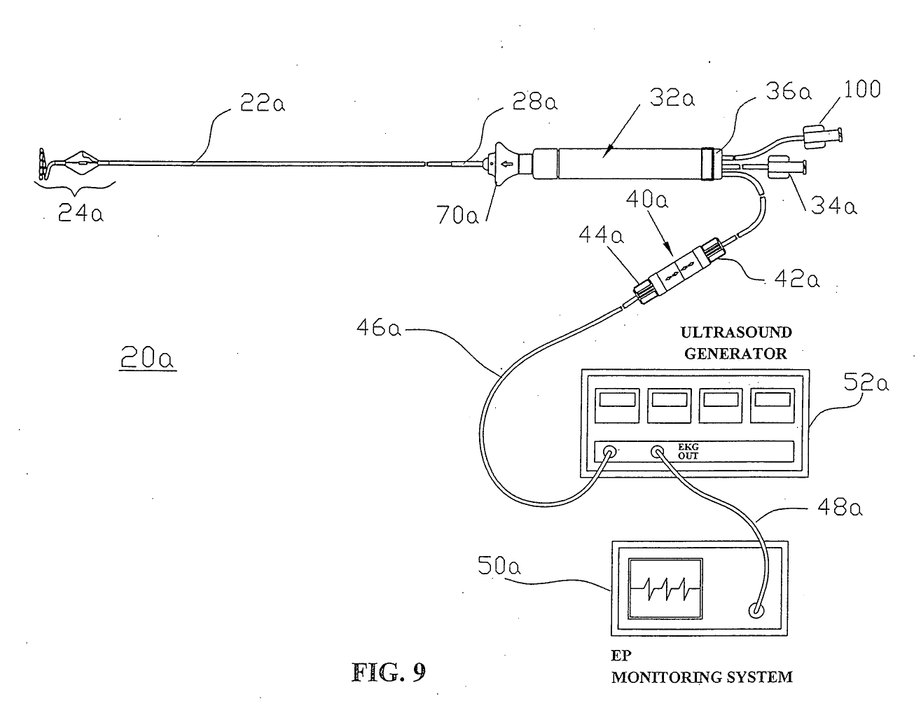

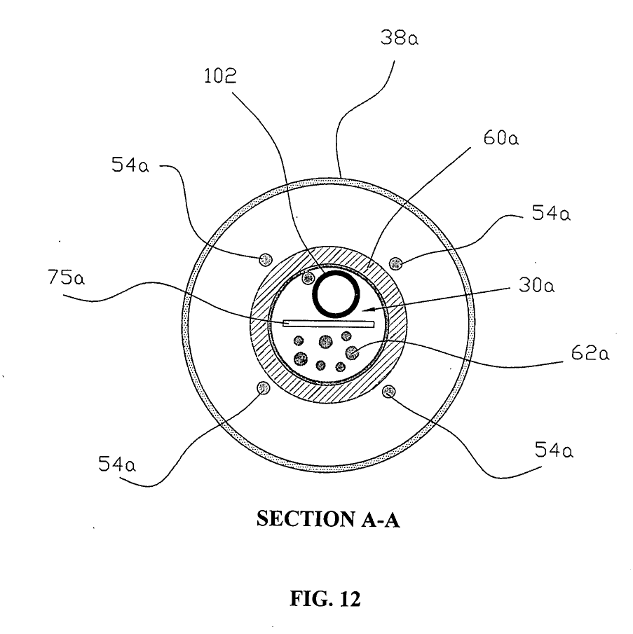

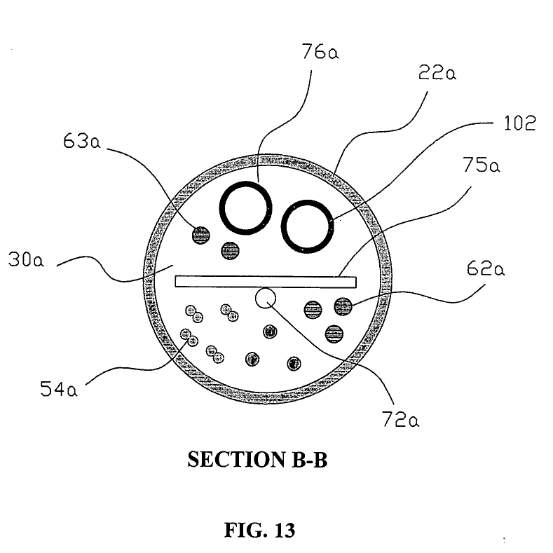

FIGS. 9-14 illustrate modifications made to the catheter system 20 of FIGS. 1-5 to allow contrast medium to be introduced while the catheter is located within the vessel ostium and the balloon 38 inflated. The catheter system 20a in FIGS. 9-14 essentially provides an additional tubing and lumen to facilitate the injection of the contrast medium. The catheter system 20 in FIGS. 1-5 did not provide an additional lumen, so the contrast medium for vessel geometry and catheter location could not be readily verified. Hence, the catheter system 20a makes it easier to verify vessel geometry and catheter location since the blood flow from within the vessel will not wash out when the contrast medium is injected due to balloon inflation.

Since the catheter system 20a merely includes modifications to the catheter system 20, the descriptions relating to the same elements and their functions will not be repeated herein. Instead, the same numerals used to designate elements in FIGS. 1-5 will be used to designate the same elements in FIGS. 9-14, except that an “a” will be added to the designations in FIGS. 9-14.

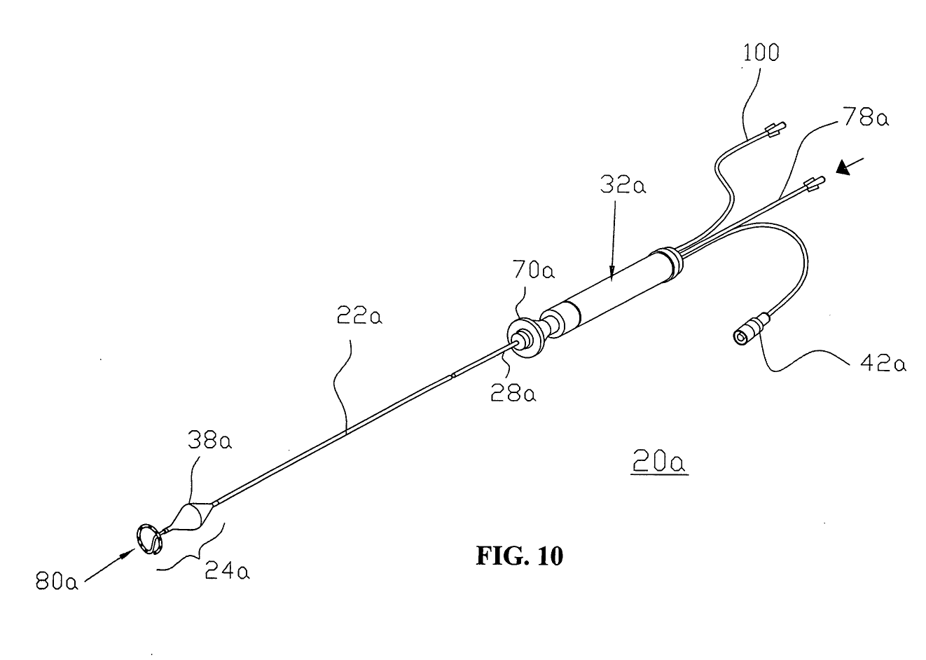

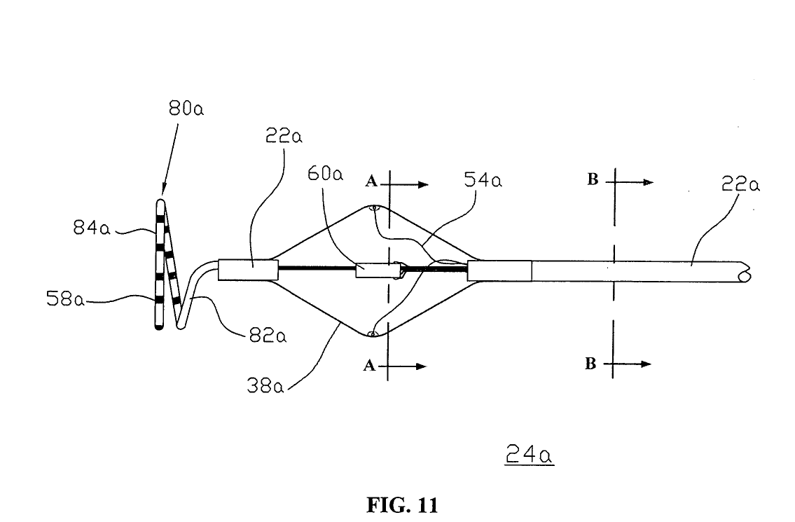

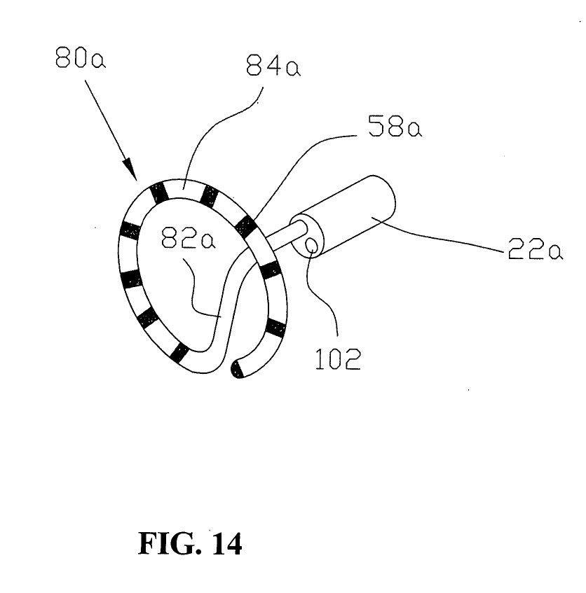

The catheter system 20a provides an additional tubing 100 that extends from the handle assembly 32a (see FIGS. 9-10). This tubing 100 is connected to a lumen 102 that extends through the shaft 22a, the transducer 60a inside the balloon 38a, and exits at the distal-most end of the shaft 22a. See FIGS. 11 and 14. The contrast medium can be injected via the tubing 100 and the lumen 102 by a syringe (not shown), and exits the catheter into the blood vessel at the location of the distal ring 80a to provide visibility of the location of the distal ring 80a and the balloon 38a. A guidewire (not shown) can be inserted into this lumen 102 to increase the mobility of the shaft 22a into branches of the main vessel.

In addition, the flat wire 75a extends in the lumen 30a from the distal section of the shaft 22a (not shown in FIGS. 9-14).

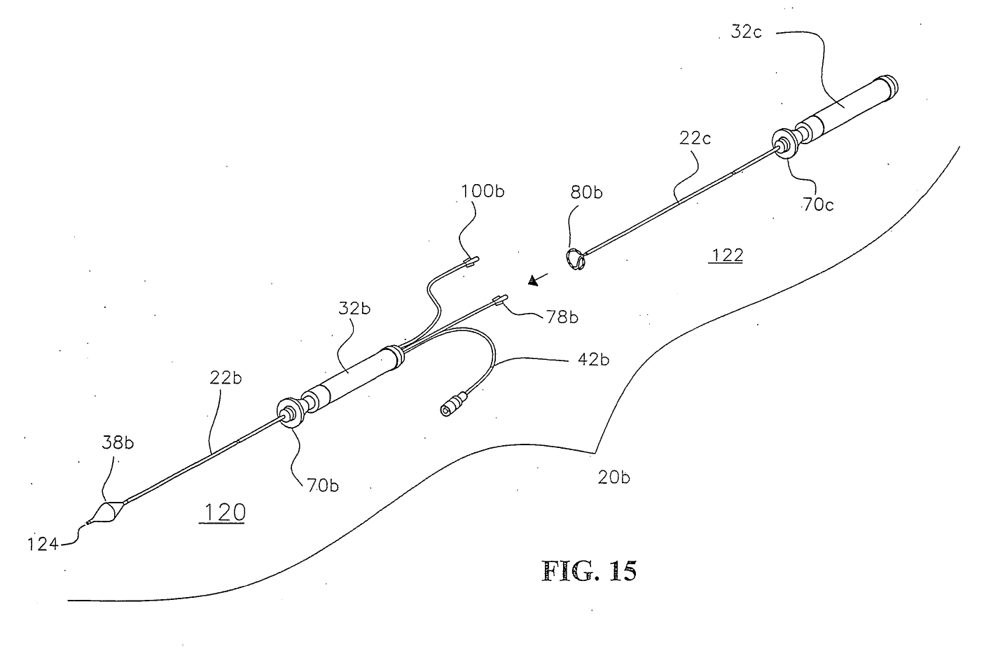

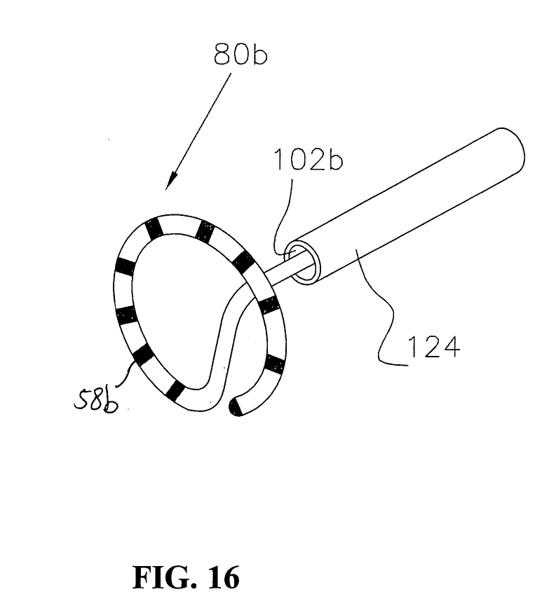

FIGS. 15-16 illustrate yet another modification that can be made to the system 20 in FIGS. 1-5. The catheter system 20b in FIGS. 15-16 is comprised of two separate catheters, a first catheter 120 that carries the balloon 38b and the transducer 60b, and a second catheter 122 that carries the distal ring 80b.

Since the catheter system 20b merely includes modifications to the catheter system 20a, the descriptions relating to the same elements and their functions will not be repeated herein. Instead, the same numerals used to designate elements in FIGS. 9-14 will be used to designate the same elements in FIGS. 15-16, except that a “b” or a “c” will be added to the designations in FIGS. 15-16. The only notable differences are (i) the catheter 120 has the same structure as the catheter 20a with the exception of the distal ring 80a, and (ii) the catheter 122 has the same structure as the catheter 120 except for the balloon 38a, the transducer 60a, and the thermocouples.

The distal ring 80b and the shaft 22c of the catheter 122 can be inserted through the lumen 102b of the catheter 120. In this regard, the distal ring 80b can progressively straightened out and drawn into the lumen 102b of the catheter 120. Thus, when confined with the catheter 120, the distal ring 80b assumes the generally linear low profile shape of the catheter 120. When the distal ring 80b exits the distal-most end 124 of the catheter 120 (see FIG. 16), the distal ring 80b is uncovered and its shape memory (e.g., Nitinol) will cause the distal ring 80b to re-assume its preformed generally circular shape.

The catheter 122 can also be steered so that the diameter of the distal ring 80b can be varied. This can be accomplished by providing a pulling wire (not shown, but can be the same as 72 or 72a), and then pulling the pulling wire. The catheter 120 can also be steered so that the distal end 124 can be deflected. The steering of the catheters 120, 122 can be accomplished using steering mechanisms 70b, 70c that can be the same as the steering mechanism 70 described in FIGS. 1-5.

The main lumen 30b of the catheter 120 can be used to accomodate a guidewire (not shown), and can also be used for delivering contrast medium. Therefore, the catheter system 20b does not require an additional tubing (such as 100) or lumen (such as 102) as in the catheter system 20a, although it is also possible to provide an additional tubing (such as 100) or lumen (such as 102) if such is desired.

The following illustrates one example of a possible use of the catheter system 20b. A transseptal sheath (with a dilator in the sheath lumen) is typically inserted into the patient's femoral vein and placed into the right atrium. Using a transseptal (Brockenbrough) needle, a puncture is produced in the fossa ovalis in the septal wall to provide access from the right atrium to the left atrium. The sheath is then brought inside the left atrium, the needle removed, and a guidewire is inserted through the lumen of the dilator to the target pulmonary vein or its branches. The distal opening of the dilator inside the sheath follows the guidewire to the pulmonary vein. When catheter 20a is used, the dilator and the guidewire are removed and the catheter inserted into the transseptal sheath into the pulmonary vein. When catheter 120 is used, only the dilator is removed and the lumen 102b of the distal of the catheter follows the path of the guidewire and into the target PV. Once the catheter 20a or 120 is situated in the pulmonary vein ostium, the balloon 38a or 38b is inflated until it engages the ostial wall. Contrast media is injected into the lumen 102 or 102b to visually verify the location of the transducer 60a with respect to the pulmonary vein anatomy.

For the catheter 20a, the location of the transducer 60a can be verified via contrast medium injection while the distal ring 80a records the PV potentials. This has not been possible with the conventional systems.

For the catheter system 20b, the catheter 122 is inserted through the tubing 100b and the distal ring 80b exits from the lumen 102b. The diameter of the distal ring 80b can be adjusted to fit the different sizes of the pulmonary vein. The electrodes 58b are again used to pick up the PV potentials. Once the potentials (or intracardiac signals) are recorded, the catheter 122 can be removed, and if needed, contrast medium can be injected for locating the transducer. Energy can then be delivered to perform the ablation, as described above.

While the description above refers to particular embodiments of the present invention, it will be understood that many modifications may be made without departing from the spirit thereof. The accompanying claims are intended to cover such modifications as would fall within the true scope and spirit of the present invention.

1. A catheter for sensing electrical events about a selected annulus region of the heart and for treating tissue in the selected annulus region, comprising:
a handle assembly;
a shaft having a proximal end coupled to the handle assembly, and a distal end, the shaft extending along an axis;
a distal ring provided at the distal end and oriented perpendicular to the axis of the shaft, the distal ring having a plurality of electrodes positioned in spaced-apart manner about the distal ring;
an ablation element positioned spaced apart from the distal ring;
means for introducing contrast medium to the distal ring.

2. The catheter of claim 1, wherein the ablation element emits energy to a radially surrounding area.

3. The catheter of claim 1, further including an expandable member covering the ablation member.

4. The catheter of claim 1, wherein the distal ring has a diameter that is greater than the fully expanded diameter of the expandable member.

5. The catheter of claim 3, wherein the expandable member defines an interior space that is filled with a fluid.

6. The catheter of claim 1, wherein the shaft has a main lumen, and further including a plurality of wires that are coupled to the plurality of electrodes and extending through the main lumen.

7. The catheter of claim 2, wherein the shaft has a main lumen, and further including a plurality of wires that are coupled to the ablation element and extending through the main lumen.

8. The catheter of claim 1, wherein the shaft has a main lumen, and further including a steering mechanism that includes a steering wire extending through the main lumen to the distal end.

9. The catheter of claim 1, wherein the shaft has a main lumen, and further including a plurality of thermocouple wires that are coupled to the distal end and extending through the main lumen.

10. The catheter of claim 3, wherein the shaft has a main lumen, a second lumen for injecting contrast medium from the distal ring, and a third lumen for inflating the expandable member.

11. A system for sensing electrical events about a selected annulus region of the heart and for treating tissue in the selected annulus region, comprising:
a catheter having:
a handle assembly;
a shaft having a proximal end coupled to the handle assembly, and a distal end, the shaft extending along an axis;
a distal ring provided at the distal end and oriented perpendicular to the axis of the shaft, the distal ring having a plurality of electrodes positioned in spaced-apart manner about the distal ring;
an ablation element positioned spaced apart from the distal ring;
means for introducing contrast medium to the distal ring;
an energy source coupled to the ablation element; and
means coupled to the plurality of electrodes for processing electrical signals received from the plurality of electrodes.

12. The system of claim 11, wherein the processing means includes a processor and a monitor.

13. The system of claim 11, wherein the ablation element emits energy to a radially surrounding area.

14. The system of claim 11, further including an expandable member covering the ablation member.

15. The system of claim 11, wherein the distal ring has a diameter that is greater than the fully expanded diameter of the expandable member.

16. A method of ablating tissue in a body cavity, comprising:
providing a catheter having a shaft having a proximal end and a distal end, with the distal end of the shaft having a mapping element and an ablation element that is separate and spaced apart from the mapping element;
positioning the mapping element at the desired treatment location in the body cavity, including injecting contrast medium to the distal ring of the catheter;
mapping distally to the desired treatment location; and
ablating the desired treatment location.

17. The method of claim 16, wherein the step of providing a catheter includes:
providing the mapping element in the form of a distal ring that is oriented perpendicular to the shaft and having a plurality of electrodes positioned in spaced-apart manner about the distal ring; and
providing the ablation element in the form of a transducer housed inside an expandable element.

18. The method of claim 17, further including:
expanding the expandable element to a maximum diameter that is less than the smallest diameter of the distal ring.

19. The method of claim 17, further including:
anchoring the distal ring in the body cavity.

20. The method of claim 16, wherein the step of ablating the desired treatment location includes emitting energy to the desired treatment location.

21. A system for sensing electrical events about a selected annulus region of the heart and for treating tissue in the selected annulus region, comprising:
(a) a first catheter having:
a handle assembly;
a shaft having a proximal end coupled to the handle assembly, and a distal end, the shaft extending along an axis; and
an ablation element provided adjacent the distal end of the shaft;
(b) a second catheter having:
a handle assembly;
a shaft having a proximal end coupled to the handle assembly of the second catheter, and a distal end, the shaft of the second catheter extending along an axis; and
a distal ring provided at the distal end of the shaft of the second catheter and oriented perpendicular to the axis of the shaft of the second catheter, the distal ring having a plurality of electrodes positioned in spaced-apart manner about the distal ring of the second catheter;
(c) an energy source coupled to the ablation element; and
(d) means coupled to the plurality of electrodes for processing electrical signals received from the plurality of electrodes.

22. A catheter for sensing electrical events about a selected annulus region of the heart and for treating tissue in the selected annulus region, comprising:
a handle assembly;
a shaft having a proximal end coupled to the handle assembly, and a distal end, the shaft extending along an axis;
a distal ring provided at the distal end and oriented perpendicular to the axis of the shaft, the distal ring having a plurality of electrodes positioned in spaced-apart manner about the distal ring;
an ablation element positioned spaced apart from the distal ring;
means for introducing a guidewire to the distal ring.
